(19) United States
(12) Patent Application Publication
(10) Pub. No.: US 2007/0197867 A1
(43) Pub. Date: Aug. 23, 2007

(54) INFLATABLE GUIDE DEVICE

(75) Inventor: Chul Hi PARK, Los Altos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Chul Hi Park, Los Altos, CA (US)

(21) Appl. No.: 11/676,506

(22) Filed: Feb. 19, 2007

Related U.S. Application Data
(60) Provisional application No. 60/775,947, filed on Feb. 22, 2006.

(51) Int. Cl. A61B 1/00

(52) U.S. Cl. 600/114; 600/115; 600/146

(57) ABSTRACT

A guide device for an endoscope includes a plurality of tubular members that are configured to be inflated and collapsed. The plurality of tubular members are arranged and joined to form a composite tubular configuration defining a lumen. The guide device is configured to receive an endoscope within the lumen and guide the endoscope along a path within an organ.

### BACKGROUND OF THE INVENTION

This invention relates to an assist device in the insertion into a tortuous passage and maneuver therein of an endoscope and method of use. More particularly, it relates to a device and method to facilitate the insertion and maneuver of a flexible endoscope into a tortuous bodily passage, such as the colon, for examination and treatment.

An endoscope is a medical instrument of an elongate tubular shape for visualizing the interior of a patient's body. Endoscopes can be used for a variety of different diagnostic and interventional procedures, including colonoscopy, sigmoidoscopy, bronchoscopy, thoracoscopy, laparoscopy and video endoscopy.

Colonoscopy is a medical procedure in which a flexible endoscope, or colonoscope, is inserted into the patient's colon for diagnostic examination and/or surgical treatment of the colon. A standard colonoscope is typically 135-185 cm in length and 12-19 mm in diameter, and includes a fiber-optic imaging bundle or a miniature camera located at the instrument's tip, illumination fibers, one or two instrument channels that may also be used for insufflation or irrigation, air and water channels, and vacuum channels. A colonoscope is sufficiently stiff so that it does not buckle when it is pushed from proximal end during insertion yet flexible enough to be maneuvered through the tortuous lumen of the colon. The final six inches of the distal end of a colonoscope is usually controllable by operating controls at the end of the colonoscope outside the patient.

The most commonly used procedure for examining the colon is first to insert a colonoscope as far as is desired while inspecting as the colonoscope advances. A detailed examination of the colon is made as the colonoscope is withdrawn. To examine the entire colon, the colonoscope is inserted through the anus into the rectum, and then advanced through the sigmoid colon into the descending colon. The colonoscope then passes through the left colic flexure (the splenic flexure) into the transverse colon, and then through the right colic flexure (the hepatic flexure). The colonoscope next passes through the ascending colon and finally reaches the cecum.

To insert a colonoscope the instrument is grasped at a point outside the body near the anus. The operator pushes the colonoscope inwards and also manipulates the colonoscope so that the inward tip is aimed in the required direction. The colon is difficult to intubate with a flexible colonoscope due to the flexible nature of the colonoscope shaft and the floppy nature of the colon. In some people the sigmoid colon can be very long and is unfixed, except by its mesentery, and so can be extremely difficult to intubate due to its predisposition to form loops when a colonoscope is pushed through it. Some anatomical landmarks, such as the rectosigmoidal junction, the splenic flexure and the hepatic flexure, are difficult to pass through simply because of their tortuous nature. Problems traversing these areas are exacerbated by looping of the colonoscope in the sigmoid colon and subsequent stretching of the sigmoid colon causing discomfort for the patient undergoing the procedure.

The insertion of endoscope can be uncomfortable for the patient. However, it is generally undesirable to anaesthetize the patient because the patient will be unable to provide feedback to the endoscopist regarding the presence or intensity of pain, which will provide the endoscopist with a helpful indication that the endoscope has been misdirected. There is a risk of perforation of the colon wall by the advancing colonoscope during insertion even when performed by skilled and experienced endoscopist.

Quite often in a colonoscopy procedure for colorectal cancer screening, the insertion of the colonoscope all the way to cecum takes a lot longer than the withdrawal during which actual inspection of the colon occurs. In some cases, screening, diagnostic or therapeutic colonoscopy procedures are abandoned even before the tip of the endoscope reaches the desired depth in the colon because the patient complains intolerable level of pain and further advancement of the endoscope is simply impractical. This leaves diagnosis or treatment of the colon incomplete because a portion of the colon beyond the tip of the endoscope will not have been examined or treated.

From a purely mechanical standpoint, one of the difficulties in intubating the colon with an endoscope originates from the fact that every colon is uniquely shaped. No single standardized approach to intubating the colon could be formulated. Complicating the problem is the fact that the colon is too distensible and the colon wall is too frail to mechanically support the winding movement of an endoscope advancing through colon's tortuousness. If the colon and colon wall were much more rigid and tougher, respectively, than they are a force stronger than normally allowable could be applied to advance an endoscope without causing undue discomfort to patient or injury to the colon wall. As a result colonoscopy procedures would become shorter and easier and the success rate would improve.

One solution to these and other difficulties of colonoscopy would be to provide a means for guiding and supporting the movement of an endoscope inside the colon with suitable mechanical characteristics, which could be readily deployed in and removed from the colon. Preferably, this means would also have to be easy to operate so as not to add another layer of complexity to already complicated procedure. Because only access to the colon is the anus, which would be constricted by the shaft of endoscope, additionally introducing a conventional guide device into the colon would be nontrivial.

Guide devices satisfying these and other requirements are described in U.S. patent application Ser. No. 11/213,285 and U.S. Prov. App. No. 60/776,597, assigned to the same inventor and filed on the same day as the present application, the disclosures of which are hereby incorporated by reference in their entirety. Although relatively uncomplicated configurations of the devices associated therewith are advantageous in the production and use, they may limit the range of mechanical characteristics of the guide devices. Guide devices with configurations that allow fine tuning of their mechanical characteristics will be more readily adaptable to a wide range of anatomical variations often found in tortuous bodily passage such as the colon.

### BRIEF SUMMARY OF THE INVENTION

An inflatable guide device and method of use for assisting insertion into a tortuous passage, for example, the colon, and maneuver therein of an endoscope is described herein. Although the embodiments described in this specification specifically refers to the colon and colonoscopy procedure, the scope of their applicability is not limited to any particular bodily organ or procedure. The terms “section” and “portion” are used interchangeably.

The present guide device generally comprises a plurality of continuous, collapsible tubular members pre-configured, arranged and joined together so as to form a composite tubular configuration with a lumen or a passage, through which an endoscope can be advanced. The present guide device is passively deployed in the colon by advancing action of the endoscope.

In one embodiment, the tubular members comprising the present guide device are made of continuous tubes configured to take a predetermined shape when inflated above an ambient pressure. Preferably, in deflated state, component tubular members comprising the tubular members are soft enough to be pleated into a form with a low profile. The external surfaces of the tubular members are coated with low friction material such as PTFE or hydrophilic material. The distal ends of the tubular members are sealed in airtight manner and the proximal ends are attached to the distal ends of the internal supply tubes of a supply tube assembly. There is provided a conduit or conduits in the supply tube assembly through which a fluid may pass to and from between a pressure controller unit external to the colon and the tubular members to control the internal pressure thereof. Preferably, the fluid is a gas (e.g., air, nitrogen, or carbon dioxide). Alternatively, it may be a low viscosity liquid (e.g., water or saline solution). A tubular member can have varying transverse dimensions along the length thereof, which may be, e.g., between 2 mm and 15 mm. The length of the tubular members may be chosen long enough for the construction of the present guide device.

The present guide device is constructed by joining together a plurality of pre-configured tubular members in predetermined arrangement using predetermined methods. Any number, e.g. between 2 and 10, of the component tubular members may be employed to construct a tubular member assembly comprising the present guide device. With the component tubular members in collapsed state the present guide device can be folded and put into a low profile form suitable for stowage in low profile housing. When the component tubular members are fully inflated the present guide device takes on a predetermined configuration generally with a lumen or a passage through which the shaft of endoscope can pass separated from the colon wall where the present guide device is deployed. Preferably, the transverse dimension of the lumen or the passage is just large enough, e.g., between 5 mm and 20 mm to allow the endoscope in use to pass freely.

The configuration of the present guide device, preferably, includes structural components, which may be referred to as a support section and a shaped section. The support section has a substantially straight configuration that is resistant to bending under a transverse load such as those applied by the shaft of advancing endoscope being guided by the lumen or the passage therein. It provides columnar strength that prevents the shaft of flexible endoscope from bending excessively. In the shaped section, the tubular members comprising the present guide device are configured to include curved portions of varying shapes and degrees of curvature to facilitate omni-directional bending of the shaped section while maintaining the patency of the lumen or the passage therein. With its high degree of flexibility the shaped section helps the present guide device closely follow the contour of sharp bends in the colon.

Preferably, the structure of the present guide device may be composed of support and shaped sections arranged in an alternating manner. Alternatively, the present guide device may be composed only of a support section or a shaped section. Preferably, the overall length of the guide device is predetermined not to exceed the maximum depth of insertion of endoscope and may be, e.g., between 20 cm and 180 cm.

In use, the tubular members comprising the present guide device, preferably, are introduced into the colon in a collapsed state stowed in low profile housing. The housing may be detachably mounted, tightly encircling the shaft of endoscope near the distal end to facilitate the passage of the guide device housing and endoscope assembly through the constricted anus. Alternatively, the housing may be of a stand-alone type to be first introduced into the colon preceding the endoscope and later engaged by the shaft of endoscope near the distal end inside the colon.

Preferably, the tubular members are sequentially released from the housing as the endoscope advances through the colon and immediately inflated by the fluid supplied through the conduit in the supply tube assembly to form a stiffened configuration, which conforms to the contour of the colon. The inflated portions of the tubular members preferably remain stationary with respect to the colon wall while allowing the endoscope to move freely through the lumen or the passage of the present guide device without exerting undue force on the colon wall. At the end of use the tubular members may be deflated into a collapsed state and withdrawn from the colon.

The housing for the tubular members may be made of a pair of soft, thin polymer film materials. The collapsed portions of the tubular members are stowed in the housing sandwiched by the two polymer films. Preferably, the overall thickness of the housing including the stowed portions of the tubular members is as thin or of a low profile as practically possible to minimize patient's discomfort during its passage through constricted anus piggybacking on the shaft of endoscope or by itself preceding the introduction of the endoscope. Preferably, the proximal ends of the tubular members comprising the present guide device are attached to and held in position by the internal supply tubes. As the endoscope advances with the housing attached to the shaft near the distal end, the tubular members stowed in the housing are pulled sequentially from the proximal end thereof and deployed.

When the depth of insertion of endoscope exceeds the overall length of the present guide device the distal ends of the tubular members leave the housing completely. On withdrawal of endoscope, the string fixed to the distal ends of the tubular members may be pulled from outside the colon along with withdrawing endoscope, sequentially deflating the tubular members starting from the distal ends. Alternatively, the distal ends of the tubular members may be made to remain tethered to the distal end of endoscope after leaving the housing so that it could be sequentially deflated in step with withdrawing endoscope. Preferably, a relief valve attached to the supply tube assembly set at predetermined point keeps the portions of the tubular members, not crumpled by withdrawing action, inflated so that it could continue to guide the endoscope throughout the withdrawal process.

When the endoscope reaches the rectum and the tubular members are fully deflated, the endoscope may be first withdrawn from the colon to allow easy removal of the crumpled tubular members through the anus. After removing the tubular members, the endoscope may be inserted back into the colon to examine the region of rectum previously blocked from view by the crumpled tubular members. Alternatively, rectum may be examined prior to commencing the advancement of endoscope soon after the endoscope is introduced into rectum. The present guide device may also be left in its fully inflated configuration for repeated insertion and removal of endoscope during procedures requiring multiple accesses to a site of interest inside the colon.

There are numerous advantages of using the present guide device as an aid for the insertion into a tortuous passage and maneuver therein of an endoscope. For example, the present guide device acts as a stationary and rigid track with a lower friction coefficient than the colon wall on which the shaft of endoscope travels, thereby reducing force needed to advance or withdraw the endoscope. The colon wall is largely shielded from the moving shaft of endoscope by the inflated tubular members of the present guide device, which spreads the force exerted by the shaft of endoscope over a wide area of the colon wall, substantially reducing possibility of injuries to the mucosal tissue thereof. The structural rigidity inherent to the present guide device effectively resists being stretched or compressed by the force exerted by the flexible shaft of endoscope, which helps the colon remain close to its natural shape during the insertion and withdrawal of endoscope.

The present guide device also minimizes the stretching of the sigmoid colon and mesentery attached thereto even when the endoscope forms a loop in the sigmoid colon, which is known to be closely associated with occurrence of patient discomfort. The present guide device also allows an endoscopist to apply pushing force that is stronger than that normally exerted by an endoscopist in a typical colonoscopy procedure wherein the upper limit on the magnitude of force is generally determined by the level of discomfort tolerable by the patient or the susceptibility of the colon wall to injuries caused by parts of advancing endoscope. The present guide device helps reduce the tendency of flexible endoscope to bend in long linear parts of the colon, which improves the transfer of pushing force from the proximal to distal ends of the endoscope. The present guide device facilitates multiple insertion and withdrawal of endoscope in a single colonoscopy procedure. Other benefits and advantages of the guide device would be appreciated by those skilled on the art based on the description provided herein.

In one embodiment a guide device for an endoscope includes a plurality of tubular members that are configured to be inflated and collapsed; a plurality of support portions; and a plurality of shaped portions. The plurality of support portions and the plurality of shaped portions are arranged in an alternating manner and together comprise the tubular members. Alternatively, the tubular members may be composed only of a support portion or a shaped portion. The guide device is configured to receive an endoscope and guide the endoscope along a path within an organ.

In yet another embodiment, a device for viewing inside of an organ includes an endoscope for inserting and viewing inside of an organ; and a guide component provided around the endoscope to facilitate insertion of endoscope within the organ. The guide component is configured to be expanded and collapsed. The guide component includes a plurality of support sections to provide columnar strength, and a plurality of shaped sections to provide flexibility to the guide component. The plurality of support sections and the plurality of shaped sections are arranged in an alternating manner. Alternatively, the present guide device may be composed only of a support section or a shaped section. The support sections are substantially straight and may be referred to as a straight portion. The shaped section includes a looped or curved section and may be referred to as a looped portion. The guide component is configured to be a single-use component that is to be discarded after a single use.

### BRIEF DESCRIPTION OF THE DRAWINGS

The following exemplary figures are provided to supplement the description below and more clearly describe the invention. In the figures, like elements are generally designated with the same reference numeral for illustrative convenience and should not be used to limit the scope of the present invention.

FIG. 1 is a perspective view of a configuration of a portion of a component tubular member of a tubular member assembly comprising a guide device in inflated state wherein straight and looped sections are alternately arranged according to one embodiment of the present invention.

FIG. 2A is a perspective view of a configuration of a portion of a support portion of a tubular member assembly, having a plurality of component tubular members in an exemplary configuration as shown in FIG. 1, comprising a guide device in inflated state according to one embodiment of the present invention.

FIG. 2B is a perspective view of a configuration of a portion of a shaped portion of a tubular member assembly, corresponding to a support portion of the tubular member assembly in an exemplary configuration as shown in FIG. 2A, comprising a guide device in inflated state according to one embodiment of the present invention.

FIG. 3A is a perspective view of another configuration of a portion of a support portion of a tubular member assembly, having a plurality of component tubular members in an exemplary configuration as shown in FIG. 1, comprising a guide device in inflated state according to one embodiment of the present invention.

FIG. 3B is a perspective view of another configuration of a portion of a shaped portion of a tubular member assembly, corresponding to a support portion of the tubular member assembly in an exemplary configuration as shown in FIG. 3A, comprising a guide device in inflated state according to one embodiment of the present invention.

FIG. 4A is a perspective view of yet another configuration of a portion of a support portion of a tubular member assembly, having a plurality of component tubular members in an exemplary configuration as shown in FIG. 1, comprising a guide device in inflated state according to one embodiment of the present invention.

FIG. 4B is a perspective view of yet another configuration of a portion of a shaped portion of a tubular member assembly, corresponding to a support portion of the tubular member assembly in an exemplary configuration as shown in FIG. 4A, comprising a guide device in inflated state according to one embodiment of the present invention.

FIG. 5A is a perspective view of a configuration of a portion of a support portion of a tubular member assembly, having a plurality of spiral-shaped component tubular members, comprising a guide device in inflated state according to one embodiment of the present invention.

FIG. 5B is a perspective view of a configuration of a portion of a shaped portion of a tubular member assembly, corresponding to a support portion of the tubular member assembly in an exemplary configuration as shown in FIG. 5A and having a plurality of spiral-shaped component tubular members, comprising a guide device in inflated state according to one embodiment of the present invention.

FIG. 6A is a perspective view of a configuration of a portion of a component tubular member in inflated state, which can be used to construct the support portion of a tubular member assembly in an exemplary configuration as shown in FIG. 5A comprising a guide device according to one embodiment of the present invention.

FIG. 6B is a perspective view of portions of a plurality of component tubular members as shown in FIG. 6A in inflated state showing in detail how they may be joined with one another to construct a part of the support portion of a tubular member assembly in an exemplary configuration as shown in FIG. 5A comprising a guide device according to one embodiment of the present invention.

FIG. 7A is a perspective view of an exemplary configuration of the supply tube assembly according to one embodiment of the present invention.

FIG. 7B is a perspective view of another exemplary configuration of the supply tube assembly according to one embodiment of the present invention.

FIG. 8 is a perspective view of an exemplary embodiment of the housing for the tubular member mounted tightly encircling the shaft of an endoscope ready to be introduced into the colon according to one embodiment of the present invention.

FIG. 9 is a schematic representation showing an endoscope and a guide device partially deployed in the colon according to one embodiment of the present invention.

### DETAILED DESCRIPTION OF THE INVENTION

Embodiments of the present invention relate to a guide device capable of assisting the insertion and maneuver of an endoscope and reducing patient's discomfort and possibility of injury to the wall during endoscopic examination and treatment of the lower gastrointestinal tract or other bodily passages.

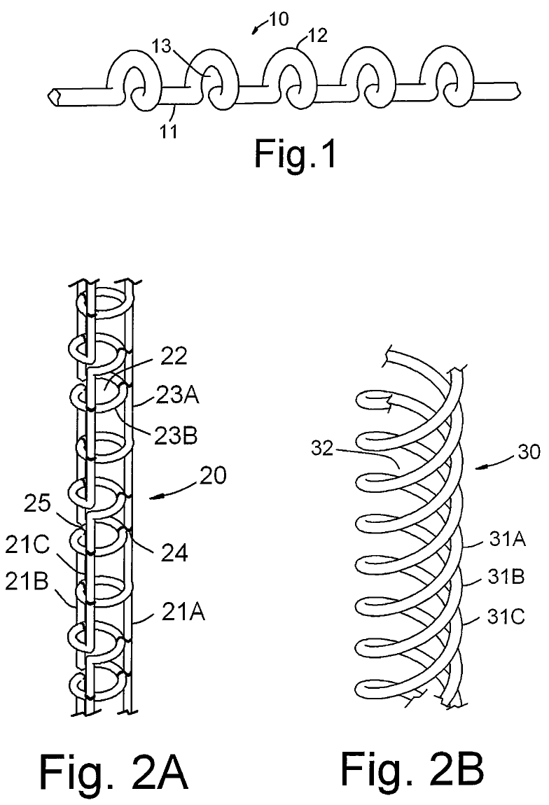

Referring to FIG. 1, an exemplary configuration of a portion of a component tubular member 10 of a tubular member assembly consisting of a guide device according to one embodiment of the present invention is shown in inflated state. Tubular member 10 generally comprises a continuous, collapsible tube, having a distal end and a proximal end, including straight 11 and looped sections 12 disposed in predetermined positions along the length thereof. An endoscope (not shown) is guided by the looped sections 12 through openings thereof 13, which keep moving shaft of endoscope from making contact with the colon wall throughout insertion process. The distal end of the tubular member is sealingly closed in the present embodiment.

The tube may be made of thin yet high tensile modulus, biocompatible polymer film material, preferably, with low frictional coefficient. Suitable construction materials may be, for example, polyethylene terephthalate (PET), polypropylene, polyamide (Nylon), and polyimide (Kapton). A suitable low friction coating (e.g., polytetrafluoroethylene (PTFE) or hydrophilic polymer) may be applied to the outer surface of the tube. Any suitable method may be employed to construct the shaped tubular member, for example, heat setting of a straight thermoplastic polymer tube into a desired shape or blow molding of a preconfigured thermoplastic polymer tube. In the present implementation, the wall thickness of the tube is no more than 0.1 mm and preferably, thinner than 0.05 mm. The thickness may vary according to application. Although these materials do not possess any structural qualities in film form, it is well known to those skilled in the art that pressure vessels of, for example, tubular or toroidal shapes constructed using thin film materials of high tensile modulus exhibit high enough structural strength for many practical applications when inflated even under moderate internal pressure. In use the internal pressure of the tubular member may be between 0.2 atmosphere and 8 atmosphere above ambient pressure. In one implementation, the internal pressure of the tubular member is between 0.3 atmosphere and 3 atmosphere above ambient pressure. In another embodiment, the internal pressure of the tubular member is more than 8 atmosphere above ambient pressure.

Still referring to FIG. 1, straight sections 11 generally provide columnar strength whereas looped sections 12 provide flexibility to tubular member 10. Due to the high tensile modulus of construction material and the configuration, tubular member 10 in inflated state is highly resistive to stretching and compression. When inflated, the mechanical characteristics, such as flexural, compressional and torsional strengths, of a tube made of polymer film material, such as those listed previously, is closely correlated with the ratio of length to transverse dimension of the tubular member. By strategically arranging straight and looped sections of suitably chosen lengths and shapes, respectively, a tubular member section with fairly specific mechanical characteristics may be constructed.

Looped sections 12 of the component tubular member 10 of a given embodiment are generally more flexible lengthwise than straight sections. The positions of looped sections 12 along the length of the tubular member 10 may be adjusted to provide different degrees of flexibility at different sections of the tubular member 10. In an exemplary configuration, several looped sections 12 may be successively joined to produce a spiral configuration, which could fit well around the sharp bends often found in the colon due to its high degree of flexibility. In another exemplary configuration, looped sections 12 may be disposed to alternate with straight sections 11 of predetermined length to make a section of the tubular member 10 more suitable for firmly supporting linear parts of the colon. The straight section 11 may be of any suitable length (e.g., between 20 mm and 60 mm or between 30 mm and 50 mm) when fully inflated. In one implementation the length of straight section 11 is 40 mm. Other arrangements may provide a portion of the tubular member 10 with flexibility somewhere between those of preceding two arrangements.

Looped sections 12 may be of various shapes and sizes. Preferably, the overall dimension of a looped section 12 is predetermined to minimize the amount material needed for construction. Preferably, the size of the opening 13 of a loop is just large enough (e.g., 10 mm and 20 mm or between 13 mm and 17 mm) to allow the endoscope in use to pass freely. In one implementation, the size of the opening 13 is 15 mm. Preferably, the external dimension of a looped section 12 is large enough to provide necessary structural strength to the looped section 12 (e.g., between 15 mm and 40 mm when fully inflated or between 20 mm and 35 mm when fully inflated). In one implementation, the looped section 12 is 25 mm long when fully inflated. Preferably, looped sections 12 with external dimensions large enough to allow the outer side thereof to frictionally engage the colon wall are strategically disposed at predetermined positions to reduce sliding of the colon with respect to the tubular member 10. The ratio of length to transverse dimension of a section of the tubular member 10 may also be adjusted to effect different degree of flexibility.

The cross-sectional profile of the tubular member 10 may be a circle, a square, a rectangle, an ellipse, or of any other irregular shape. The cross sectional profile has a measurable influence on the flexibility of the thin walled tubular member 10, which may be advantageously applied to fine-tune the flexibility of different sections of the tubular member 10. Preferably, the tubular member 10 may have several different cross-sectional profiles along the length thereof. Preferably, the overall length of the guide device is predetermined not to exceed the maximum depth of insertion of endoscope in a procedure. The dimension of the cross-sectional profile is between 5 mm and 20 mm in the present embodiment, but may be of different dimension according to application. In one implementation, the dimension of the cross-sectional profile is between 8 mm and 15 mm. In another implementation, the dimension of the cross-sectional profile is approximately 10 mm.

FIG. 2A shows an exemplary configuration of a portion of a support portion 20 of a tubular member assembly comprising a plurality of component tubular members 21A, 21B, 21C in an exemplary configuration as shown in FIG. 1 comprising a guide device in inflated state according to one embodiment of the present invention. Component tubular members 21A, 21B, 21C are arranged in support portion 20 of a tubular member assembly in such angular relations with one another that the straight sections 23A are positioned around the circumference of the looped sections 23B at substantially equal angular displacement with one another and looped sections 23B are substantially evenly spaced along the length of support portion 20. Straight sections 23A of component tubular members 21A, 21B, 21C are positioned in close proximity to outer rims of and are joined with looped sections 23B with predetermined methods. Suitable joining methods, for example, may be fastening straight sections 23A and looped sections 23B together using a low profile fastener 24 of predetermined material, for example, a tie string made of high tensile strength polymer, for example, PET. Alternatively, a biocompatible adhesive may be employed with or without the fastener 24.

Support portion 20 of a tubular member assembly thus formed has a much higher stiffness lengthwise than a component tubular member 10 in an exemplary configuration as shown in FIG. 1 since a bending load on one side of support portion 20 is opposed by a plurality of straight sections 23A positioned diagonally from where the load is applied. To further strengthen support portion 20 the gaps 25 between the two end points of looped sections 23B may be joined with each other with similar joining methods explained above.

The shaft of an endoscope (not shown) is guided by and advances while confined within the lumen 22, defined by the series of openings of looped sections 23B of component tubular members 21A, 21B, 21C, which keep moving shaft of endoscope from making contact with the colon wall throughout insertion process. This configuration may be suitable for guiding an endoscope in linear parts of the colon, which helps a flexible endoscope to remain straight under the bending load of two opposing forces between distal and proximal ends.

Referring FIG. 2B a perspective view of a configuration of a portion of a shaped portion 30 of a tubular member assembly is shown, which is complementary to a support portion 20 of the tubular member assembly in an exemplary configuration as shown in FIG. 2A, comprising a guide device in inflated state according to one embodiment of the present invention. In one implementation shaped portion 30 comprises a plurality of component tubular members 31A, 31B, 31C of substantially spiral-shape in identical rotational orientation. Alternatively, component tubular members 31A, 31B, 31C may be in counter rotational orientations with one another. Alternatively, component tubular members 31A, 31B, 31C may be in any curved shape other than spiral form. The number of component tubular members employed is chosen to be equal to that of support portion 20 as shown in FIG. 2A.

Shaped portion 30 of a tubular member assembly in an exemplary configuration as shown in FIG. 2B is much more flexible lengthwise than support section 20 shown in FIG. 2A and can be bent omni-directionally to a high angle without compromising the patency of the lumen 32. Lumen 32 is defined by the series of circumferences of curved component tubular members 31A, 31B, 31C, which keep moving shaft of endoscope from making contact with the colon wall around the bends in the colon throughout insertion process.

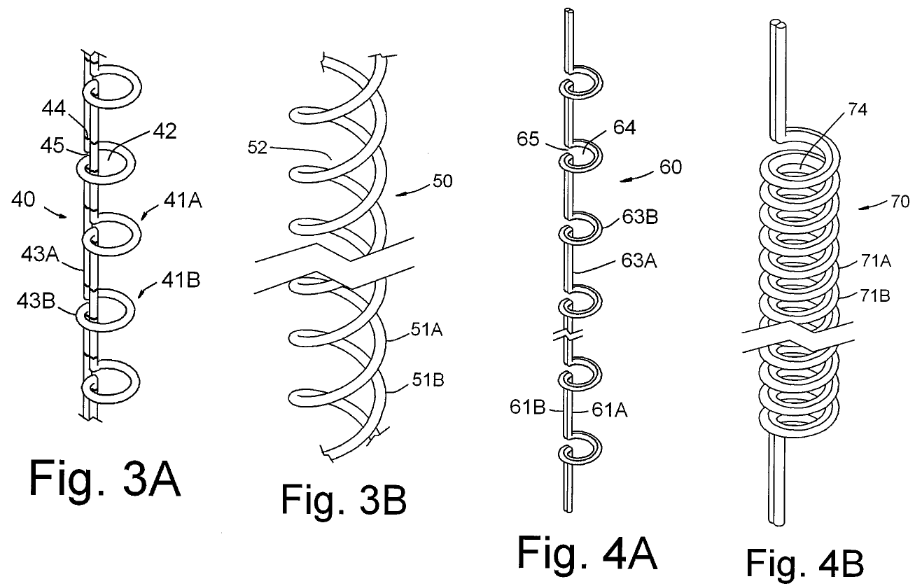

FIG. 3A shows an exemplary configuration of a portion of a support portion 40 of a tubular member assembly comprising a plurality of component tubular members 41A, 41B in an exemplary configuration as shown in FIG. 1 comprising a guide device in inflated state according to another embodiment of the present invention. Component tubular members 41A, 41B are arranged in support portion 40 of a tubular member assembly so that the straight sections 43A of component tubular members 41A, 41B are positioned in close proximity and joined with one another with predetermined methods. Suitable joining methods, for example, may be fastening straight sections 43A together using a low profile fastener 44 of predetermined material, for example, a tie string made of high tensile strength polymer, for example, PET. Alternatively, a biocompatible adhesive may be employed with or without the fastener 44.

Support portion 40 of a tubular member assembly thus formed has a much higher stiffness lengthwise than a component tubular member 10 in an exemplary configuration as shown in FIG. 1 since a bending load on one side of support portion 20 is opposed by a plurality of straight sections 43A instead of one and the gaps 45 between the two end points of looped sections 43B are prevented from opening up under a bending load to support section 40.

The shaft of an endoscope (not shown) is guided by and advances while confined within the lumen 42, defined by the series of openings of looped sections 43B of component tubular members 41A, 41B, which keep moving shaft of endoscope from making contact with the colon wall throughout insertion process. This configuration may be suitable for guiding an endoscope in linear parts of the colon, which helps a flexible endoscope to remain straight under the bending load of two opposing forces between distal and proximal ends.

Referring FIG. 3B a perspective view of a configuration of a portion of a shaped portion 50 of a tubular member assembly is shown, which is complementary to a support portion 40 of the tubular member assembly in an exemplary configuration as shown in FIG. 3A, comprising a guide device in inflated state according to one embodiment of the present invention. In one implementation shaped portion 50 comprises a plurality of component tubular members 51A, 51B of substantially spiral-shape in identical rotational orientation. Alternatively, component tubular members 51A, 51B may be in counter rotational orientations with one another. Alternatively, component tubular members 51A, 51B may be in any curved shape other than spiral form.

Shaped portion 50 of a tubular member assembly in an exemplary configuration as shown in FIG. 3B is much more flexible lengthwise than support section 40 shown in FIG. 3A and can be bent omni-directionally to a high angle without compromising the patency of the lumen 52. Lumen 52 is defined by the series of circumferences of curved component tubular members 51A, 51B which keep moving shaft of endoscope from making contact with the colon wall around the bends in the colon throughout insertion process.

FIG. 4A shows an exemplary configuration of a portion of a support portion 60 of a tubular member assembly comprising a plurality of component tubular members 61A, 61B in an exemplary configuration as shown in FIG. 1 comprising a guide device in inflated state according to yet another embodiment of the present invention. In one implementation component tubular members 61A, 61B are arranged in tandem in close proximity of and joined with one another with predetermined methods. Suitable joining methods, for example, may be fastening component tubular members 61A, 61B at predetermined locations together using a low profile fastener (not shown) of predetermined material, for example, a tie string made of high tensile strength polymer, for example, PET. Alternatively, a biocompatible adhesive may be employed with or without the fastener.

Support portion 60 of a tubular member assembly thus formed has a much higher stiffness lengthwise than a component tubular member 10 in an exemplary configuration as shown in FIG. 1 since a bending load on one side of support portion 60 is opposed by a plurality of the straight sections 63A instead of one. To further strengthen support portion 60 the gaps 65 between the two end points of the looped sections 63B may be joined with each other with similar joining methods explained above.

The shaft of an endoscope (not shown) is guided by and advances while confined within the lumen 62, defined by the series of openings of looped sections 63B of component tubular members 61A, 61B, which keep moving shaft of endoscope from making contact with the colon wall throughout insertion process. This configuration may be suitable for guiding an endoscope in linear parts of the colon, which helps a flexible endoscope to remain straight under the bending load of two opposing forces between distal and proximal ends.

Referring FIG. 4B a perspective view of a configuration of a portion of a shaped portion 70 of a tubular member assembly is shown, which is complementary to a support portion 60 of the tubular member assembly in an exemplary configuration as shown in FIG. 4A, comprising a guide device in inflated state according to one embodiment of the present invention. In one implementation shaped portion 70 comprises a plurality of component tubular members 71A, 71B of substantially spiral-shape in identical rotational orientation in tandem position. Alternatively, shaped portion 70 may be in any other curved configuration, for example, shaped portion 50 as shown in FIG. 3B.

Shaped portion 70 of a tubular member assembly in an exemplary configuration as shown in FIG. 4B is much more flexible lengthwise than support section 60 shown in FIG. 4A and can be bent omni-directionally to a high angle without compromising the patency of the lumen 72. Lumen 72 is defined by the series of openings of looped sections of component tubular members 71A, 71B, which keep moving shaft of endoscope from making contact with the colon wall around the bends in the colon throughout insertion process.

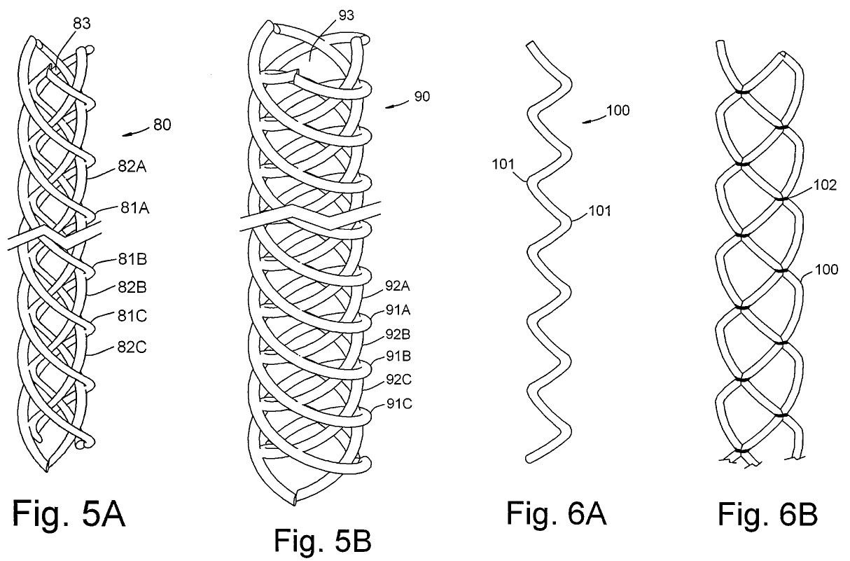

FIG. 5A shows an exemplary configuration of a portion of a support portion 80 of a tubular member assembly comprising a plurality of component tubular members 81A, 81B, 81C, 82A, 82B, 82C of substantially spiral shape comprising a guide device in inflated state according to yet another embodiment of the present invention. Component tubular members 81A, 81B, 81C are in one rotational orientation and component tubular members 82A, 82B, 82C are in counter rotational orientation. Component tubular members 81A, 81B, 81C, 82A, 82B, 82C all are of substantially equal pitch and comparable transverse dimension and are positioned in such a way that together they form a composite tubular configuration with a lumen 83 and a grid wall. Wherever a pair of component tubular members intersects an immovable joint is formed between the pair with suitable joining methods, for example, a low profile fastener (not shown) of predetermined material, for example, a tie string made of high tensile strength polymer, for example, PET. Alternatively, a biocompatible adhesive may be employed with or without the fastener.

Support portion 80 of a tubular member assembly thus formed has a much higher stiffness lengthwise than tubular member 10 in an exemplary configuration as shown in FIG. 1 since a bending load on one side of support portion 80 is opposed by a plurality of component tubular member positioned diagonally from where the load is applied.

The shaft of an endoscope (not shown) is guided by and advances while confined within the lumen 83, defined in the composite tubular configuration, which keep moving shaft of endoscope from making contact with the colon wall throughout insertion process. This configuration may be suitable for guiding an endoscope in linear parts of the colon, which helps a flexible endoscope to remain straight under the bending load of two opposing forces between distal and proximal ends.

Referring FIG. 5B a perspective view of a configuration of a portion of a shaped portion 90 of a tubular member assembly is shown, which is complementary to a support portion 80 of the tubular member assembly in an exemplary configuration as shown in FIG. 5A, comprising a guide device in inflated state according to one embodiment of the present invention. In one implementation shaped portion 90 comprises a plurality of component tubular members 91A, 91B, 91C, 92A, 92B, 92C of substantially spiral-shape, of substantially equal pitch and comparable transverse dimension, in a rotational orientations similar to component tubular members 81A, 81B, 81C, 82A, 82B, 82C as shown in FIG. 5A. Alternatively, component tubular members 91A, 91B, 91C, 92A, 92B, 92C may be have identical rotational orientations with one another. Alternatively, component tubular members 91A, 91B, 91C, 92A, 92B, 92C may be in any curved shape other than spiral form. Unlike support portion 80 as shown in FIG. 5A, component tubular members 91A, 91B, 91C, 92A, 92B, 92C are not joined together where a pair of them intersects. Component tubular members 91A, 91B, 91C, 92A, 92B, 92C also have much smaller pitch than those shown in FIG. 5A to facilitate omni-directional bending of shaped portion 90.

Shaped portion 90 of a tubular member assembly in an exemplary configuration as shown in FIG. 5B is much more flexible lengthwise than support section 80 shown in FIG. 5A and can be bent omni-directionally to a high angle without compromising the patency of the lumen 93, the grid wall of which keep moving shaft of endoscope from making contact with the colon wall around the bends in the colon throughout insertion process.

FIG. 6A shows an exemplary configuration of a portion of a component tubular member 100 in inflated state that may be used to construct a support portion of a tubular member assembly comprising a guide device in an exemplary configuration as shown in FIG. 5A according to one embodiment of the present invention. To construct the grid wall of a composite tubular configuration, a plurality of component tubular members 100 are arranged in cylindrical formation with corresponding elbows 101 of neighboring component tubular members 100 touching each other. Wherever a pair of elbows 101 meets an immovable joint 102 is formed between the pair with suitable joining methods, for example, a low profile fastener (not shown) of predetermined material, for example, a tie string made of high tensile strength polymer, for example, PET. Alternatively, a biocompatible adhesive may be employed with or without the fastener. FIG. 6B shows an exemplary configuration of partially assembled grid wall of a composite tubular configuration including component tubular members 100 and immovable joints 102 in an exemplary configuration as shown in FIG. 6A according to one embodiment of the present invention.

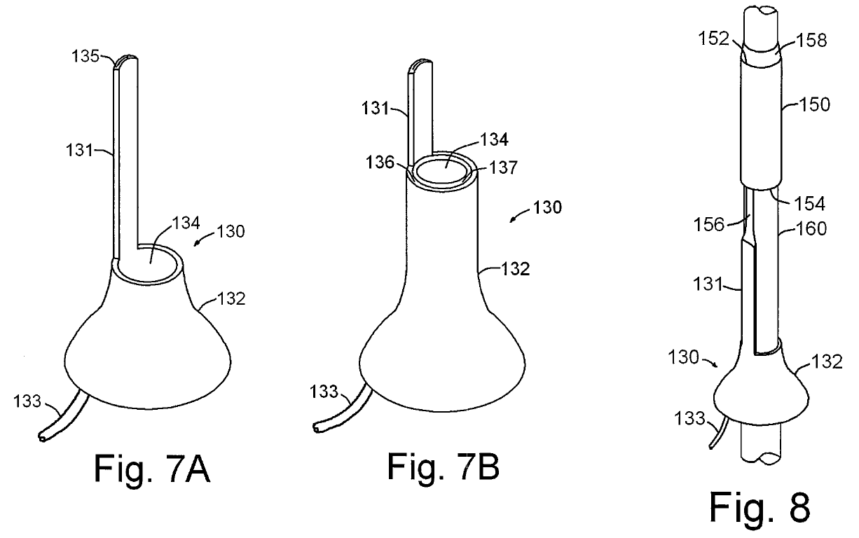

FIG. 7A is a perspective view of an exemplary configuration of a supply tube assembly 130. The supply tube assembly includes an internal 131 and an external 133 supply tubes and a funnel shaped body 132. There is provided a conduit in the funnel shaped body 32, which is in communication with the channels of internal 131 and external 133 tubes. A fluid may flow back and forth between the tubular member of the present guide device, the proximal end of which is attached fluid-tight to the internal supply tube 131 and the pressure controller unit (not shown) external to the colon, which is attached fluid-tight to the external supply tube 133. The internal supply tube 131 holds the proximal end of the tubular member immobile and causes the portion of the tubular member stowed collapsed in the housing to be sequentially released as the endoscope advances carrying the housing. Any suitable method, such as, adhesive bonding, welding, heat sealing or ultrasonic sealing may be used for joining the internal supply tube 131 and the tubular member.

Preferably, the internal supply tube 131 is of curved paddle shape to ease the introduction through the anus, piggybacking on the endoscope. Preferably, the curvature of the internal supply tube 131 is configured to allow it to be tightly fit around the shaft of endoscope. The paddle shaped internal supply tube 131 may be thin, at most 1 mm thick, and long enough for the distal end 135 to completely pass through the anal canal and reach well into the rectum. Preferably, the internal supply tube 131 along with the rest of the assembly 130 is made of pliable polymer material with low friction coefficient to facilitate the movement of endoscope as it pass through the anus sliding pressed against the internal supply tube 131. The cross-sectional area of the conduit in the internal supply tube may be varied by changing the overall width of the paddle along with the width of the conduit therein without increasing the thickness thereof. The contoured, funnel shaped body 132 remains outside of the anus rested against the patient body region surrounding the anus and acts as a fixed base for the internal supply tube 131 and the tubular member attached thereto and the external supply tube 133. The endoscope is introduced into the colon through the opening 134 in the body 132 of the assembly 130.

Alternatively, as shown in FIG. 7B, the length of the funnel shaped body 132 of the supply tube assembly 130 may be made long so that the distal end 136 thereof can be advanced into and disposed inside the rectum. In this configuration, the narrow part of the body 132 lines the anal canal completely holding the supply tube assembly 130 in position while the wide part remains outside the anus rested against the patient body region surrounding the anus. Preferably, the opening 134 of the body 132 is just large enough for endoscope to pass through unrestricted. In this arrangement, endoscope can enter or leave the colon passing through the anal canal without being constricted by the surrounding sphincter muscle. An annular seal 137 made of an elastic material, such as silicon rubber, may be disposed at the distal end 136 of the body 132 for sealingly and slidiably engaging endoscopes of various diameters.

FIG. 8 shows a perspective view of an exemplary housing 150 for the present guide device mounted tightly encircling the shaft of an endoscope 160 and an exemplary embodiment of the supply tube assembly 130 with the internal 131 and the external 133 supply tubes and the funnel shaped body 132 prior to the insertion into the colon. The deflated and pleated portion of the tubular member comprising the present guide device is stowed sandwiched between a pair of thin polymer films comprising the housing 150 while the endoscope advances along the colon. A pair of thin polymer films with material properties very similar to the ones used to construct the tubular member comprising the present guide device may be used to construct the housing 150. The distal end of the housing 152 is closed and the proximal end 154 is left open wherefrom the pleated tubular member 156 slides out.

Preferably, the annular gap between the two films, wherein the pleated portion of tubular member is stored, is at most 1 mm wide and preferably, narrower than 0.5 mm. The length of the housing 150 may be long enough for the entire guide device to be stowed deflated, for example, around 10 cm. In use, the housing 150 is mounted tightly encircling the shaft of an endoscope 160 near the distal end using, for example, a locking collar 158, which holds the housing in place. Preferably, the locking collar 158 is made of a smooth polymer film material to minimize the cross-sectional profile to facilitate the passage of endoscope and the housing through the anus.

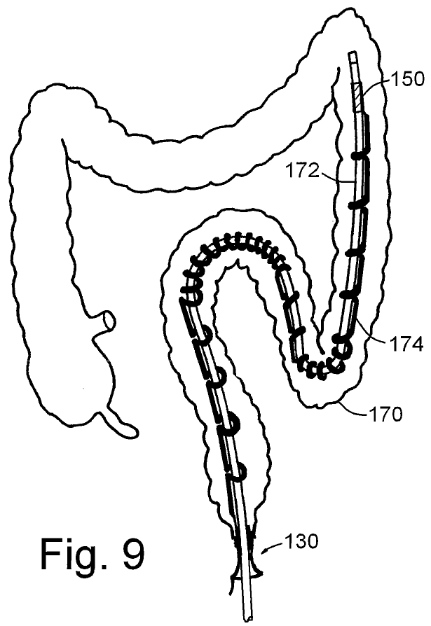

FIG. 9 schematically shows an endoscope 172 and the present guide device 174 partially inserted and deployed in the colon 170. Also shown in the figure are the housing 150, the supply tube assembly 130.

The procedure for using the present guide device is as follows. An endoscope with the guide device housing mounted tightly encircling the shaft near the distal end is inserted into the colon through the anus. As the endoscope advances along the colon, the tubular member comprising the present guide device in folded state sequentially slides out of the housing in step with the advancing endoscope. The freed portion of the tubular member is inflated into a preconfigured shape by the pressurizing fluid supplied through the conduit in the supply tube assembly. At a point where the depth of endoscope insertion exceeds the overall length of the tubular member, the tubular member becomes completely loose from the housing and assumes its full configuration.

During the withdrawal of endoscope the pull string attached to the distal end of the tubular member is pulled in step with the withdrawing endoscope to sequentially deflate the tubular member starting from the distal end. Alternatively, the distal end of the tubular member may be predisposed to remain tethered close to the housing after leaving the housing so that it could be sequentially deflated in step with the withdrawing endoscope. During withdrawal the relief valve attached to the supply tube assembly is preset to maintain a predetermined constant pressure in the tubular member. When the withdrawal process reaches the rectum the endoscope is first pulled out of the colon and subsequently the fully deflated tubular member is withdrawn. The endoscope is reinserted into the rectum to examine the area of the rectum blocked from view by the crumpled tubular member. Alternatively, the rectum may be first examined soon after its introduction into the rectum prior to commencing the advancement of endoscope. The present guide device may also be left in its fully inflated configuration for repeated insertion and withdrawal of endoscope during procedures requiring multiple accesses to a site of interest within the colon.

While preferred illustrative embodiments of the invention are described above, it will be apparent to those skilled in the art that various changes and modifications may be made therein without departing from the invention. Accordingly, the appended claims should be used to interpret the scope of the present invention.

What is claimed is:

1. A guide device for an endoscope, the guide device comprising:
a plurality of tubular members that are configured to be inflated and collapsed,
wherein the plurality of tubular members are arranged and joined to form a composite tubular configuration defining a lumen, and
wherein the guide device is configured to receive an endoscope within the lumen and guide the endoscope along a path within an organ.

2. The guide device of claim 1, wherein the guide device is configured to be a single-use component that is to be discarded after a single use.

3. The guide device of claim 1, wherein the guide device has no more than ten tubular members.

4. The guide device of claim 1, wherein the tubular members are joined using fasteners.

5. The guide device of claim 1, wherein the tubular members are joined using an adhesive.

6. The guide device of claim 2, wherein the tubular members are inflated using fluid.

7. The guide device of claim 3, wherein said fluid includes air, nitrogen, carbon dioxide, or a combination thereof.

8. The guide device of claim 3, wherein said fluid includes water, saline solution, or a combination thereof.

9. The guide device of claim 3, wherein said fluid is pressurized at a pressure between 0.2 atmosphere and 8 atmosphere.

10. The guide device of claim 1, wherein said lumen has a width in the range between 5 mm and 20 mm.

11. The guide device of claim 1, wherein said tubular members have cross sectional dimensions in the range between 5 mm and 20 mm when fully inflated.

12. The guide device of claim 1, wherein said tubular members have wall thicknesses of no more than 0.05 mm.

13. The guide device of claim 1, wherein said tubular members include polyethylene terephthalate.

14. The guide device of claim 1, wherein said tubular members include polypropylene or polyamide.

15. The guide device of claim 1, wherein said tubular members are coated with polytetrafluoroethylene or hydrophilic polymer.

16. A guide device for an endoscope, the guide device comprising:
a plurality of tubular members that are configured to be inflated and collapsed, the tubular members including a plurality of straight portions and a plurality of looped portions, wherein the straight portions and the looped portions are arranged in an alternating manner,
wherein the tubular members are arranged and joined to form a composite tubular configuration defining a lumen, and
wherein the guide device is configured to receive an endoscope in the lumen and guide the endoscope along a path within an organ.

17. The guide device of claim 15, wherein said looped portions are substantially of spiral shape.

18. A guide device for an endoscope, the guide device comprising:
a plurality of tubular members that are configured to be inflated and collapsed, the tubular members including a plurality of support portions and a plurality of shaped portions,
wherein the support portions and the shaped portions are arranged in an alternating manner,
wherein the tubular members are arranged and joined to form a composite tubular configuration defining a lumen, and
wherein the guide device is configured to receive an endoscope within the lumen and guide the endoscope along a path within an organ.
